(19) United States
(12) Patent Application Publication
(10) Pub. No.: US 2019/0328313 A1
(43) Pub. Date: Oct. 31, 2019

(54) METHOD FOR RECORDING JAW MOVEMENTS

(71) Applicant: SICAT GMBH & CO. KG, BONN (DE)

(72) Inventors: NILS HANSSEN, BONN (DE); JOCHEN KUSCH, WACHTBERG (DE); TOBIAS LEHNER, BONN (DE)

(73) Assignee: SICAT GMBH & CO. KG, BONN (DE)

(21) Appl. No.: 16/344,802

(22) PCT Filed: Oct. 16, 2017
(86) PCT No.: PCT/EP2017/076303

§ 371 Date: Apr. 25, 2019

(30) Foreign Application Priority Data
Oct. 27, 2016 (DE) 10 2016 120 583,4

(51) Int. Cl. A61B 5/00; A61B 5/11; A61C 19/045; A61C 7/12

(52) U.S. Cl. CPC A61B 5/4547; A61B 5/1114; A61C 7/12; A61B 5/682; A61C 19/045; A61B 5/4836

(57) ABSTRACT

A method for recording jaw movements of a patient includes detachably adhering dental brackets to teeth surfaces of the teeth of a lower jaw of the patient, attaching a position marker and a sensor element which detects the position marker to a head of the patient, and recording, via a jaw movement, a relative movement of the sensor element relative to the position marker. The sensor element is attached to a skull of the patient, and the position marker is attached to the lower jaw of the patient via the dental brackets, or the sensor element is attached to the lower jaw of the patient via the dental brackets, and the position marker is attached to the skull of the patient.

### CROSS REFERENCE TO PRIOR APPLICATIONS

This application is a U.S. National Phase application under 35 U.S.C. § 371 of International Application No. PCT/EP2017/076303, filed on Oct. 16, 2017 and which claims benefit to German Patent Application No. 10 2016 120 583.4, filed on Oct. 27, 2016. The International Application was published in German on May 3, 2018 as WO 2018/077643 A1 under PCT Article 21(2).

### FIELD

The present invention relates to a method and to an apparatus for recording jaw movements of a patient, wherein a sensor element and a position marker, which is detectable by the sensor element, are attached to the head of the patient. It is here in principle possible to freely select whether the sensor element is attached to the head and the position marker attached to the lower jaw, or vice versa. The jaw movements of the patient are captured and recorded by the relative movement of the sensor element in relation to the position marker.

### BACKGROUND

Such methods and apparatuses have previously been described in DE 10 2014 111 643 A1 and in DE 20 2013 001 910 U1 where bite forks are attached to the teeth adhesively or via a registration material.

DE 11 2005 000 700 T5 describes an apparatus and a method for measuring a three-dimensional jaw movement in which sensors are adhesively attached to the teeth.

DE 10 2014 225 457 A1, WO 2014/186 278 A1, WO 2007/133 422 A2 and WO 2003/096 922 A1 describe orthodontic apparatuses in which sensors are attached in or to dental brackets.

In the previously-described methods and apparatuses, temporarily rigid mechanical connections between the measurement system and fixed structures of the lower jaw, for example, the bones or the teeth, are established to measure chewing movements of a patient. The sensor element is thereby typically fixedly connected to the structures of the upper head region of the patient by placing a type of bracket holder on the patient, for example, wherein the position marker is fixedly connected to the teeth of the lower jaw. A so-called paraocclusal attachment is used to fixedly connect the position marker fixedly to the lower jaw. The paraocclusal attachment is in principle a holder that is adapted to the curvature of a patient's dental arch and is attached to the vestibular surfaces of the teeth via a cured substance or adhesive.

Disadvantageously, the attachment of the paraocclusal attachment via the cured substance or the adhesive frequently causes difficulty for the dentist because the dentist must find a compromise between sufficient support of the position marker at the teeth during the recording of the jaw movement by the measurement system and a simple detachment of the paraocclusal attachment after the measurement is finished. This requires experience and the use of suitable materials. The attachment of the paraocclusal attachment is often only inadequately successful even for experienced dentists due to the smoothness of the teeth and remainders of saliva. For patients having an overbite, the vestibular surfaces of the lower jaw teeth are frequently occluded in a terminal occlusion so that attachment of the paraocclusal attachment is difficult. Due to the weight of the measurement system which is attached to the plate of the paraocclusal attachment, high torques act on the vestibular surfaces of the teeth, which is why strong adhesion is required, in particular if the lower jaw performs rapid movements during the measurement. The adhered attachments are in practice currently disadvantageously detached all at once from the teeth as a large adhesion surface. This requires the application of a great amount of force and consequently also presents a potential injury risk to the patient.

### SUMMARY

An aspect of the present invention is to provide a method for measuring jaw movements in which a sensor element or a position marker of a measurement system is detachably attached to the teeth of the lower jaw of the patient in a simple and reliable manner. A further aspect of the present invention is to provide an apparatus for the method.

In an embodiment, the present invention provides a method for recording jaw movements of a patient which includes detachably adhering dental brackets to teeth surfaces of the teeth of a lower jaw of the patient, attaching a position marker and a sensor element which is configured to detect the position marker to a head of the patient, and recording, via a jaw movement, a relative movement of the sensor element relative to the position marker. The sensor element is attached to a skull of the patient, and the position marker is attached to the lower jaw of the patient via the dental brackets, or the sensor element is attached to the lower jaw of the patient via the dental brackets, and the position marker is attached to the skull of the patient.

### BRIEF DESCRIPTION OF THE DRAWINGS

The present invention is described in greater detail below on the basis of embodiments and of the drawings in which:

FIG. 1 shows a patient wearing a measurement apparatus according to the present invention for capturing jaw movements on the patient's head;

FIG. 2 shows a first method according to the present invention for attaching a position marker of the measurement apparatus to a patient's teeth;

FIG. 3 shows a first schematic configuration of the attachment of the position marker to treatment wires of braces for illustration purposes;

FIG. 4 shows a second schematic configuration of the attachment of the position marker to treatment wires of the braces for illustration purposes;

FIG. 5 shows a second method according to the present invention for attaching the position marker to treatment wires of a patient's braces;

FIG. 6 shows a plurality of treatment wires on a coupling element; and

FIG. 7 shows a plurality of freely-selectable attachment points on the coupling element.

### DETAILED DESCRIPTION

The main idea realized in the method according to the present invention is that dental brackets are detachably adhered to surfaces of the teeth of the lower jaw, wherein the sensor element or the position marker is attached via the dental brackets.

The dental brackets will hereby be detachably adhered to attachment points on surfaces of the teeth, wherein the sensor element or the position marker at least partially projects from the oral cavity of the patient. The dental brackets establish a fixed connection between the sensor element or the position marker of the measurement system and the teeth which withstands even large torques. Dental practices also have years of experience in using dental brackets, as they are typically used in the case of braces. Dental brackets provide good detachability from the patient's teeth after the measurement using a special tool. In the method according to the present invention, each dental bracket is advantageously individually detached gently from the teeth of the patient. Ready to use kits are readily available for reliably adhering the dental brackets onto the teeth and for removal thereof.

The attachment points can, for example, be attached mirror-symmetrically to the mirror-symmetric axis of the lower jaw. This provides for a uniform load of the lower jaw by the acting torques with the result that the dental brackets are loaded as uniformly as possible and the subjective comfort of the patient is consequently increased. The teeth for the attachment points are freely selected as appropriate. A deviation from the mirror-symmetric arrangement of the attachment points might be reasonable or necessary under certain circumstances based on dental technical aspects. It is necessary to deviate from the mirror-symmetric arrangement, for example, if the patient is missing teeth on one side of the lower jaw. In case a mirror-symmetrically present tooth is diseased, the dentist may also freely determine a different attachment point on a different tooth to spare the diseased tooth.

In a refinement of the present invention, a treatment wire can, for example, be detachably attached to the dental brackets, wherein, two dental brackets are in each case horizontally connected by way of the treatment wire, wherein the sensor element or the position marker is attached detachably between two dental brackets via the treatment wire. The sensor element or the position marker can thereby be advantageously reliably attached to the lower jaw even during an ongoing orthodontic treatment, for example, in the case of a treatment relating to braces, by using the dental brackets which have already been attached to the patient's teeth for the orthodontic treatment using the braces.

A plurality of treatment wires extend through the dental brackets where appropriate, wherein the sensor element or the position marker is attached to the plurality of treatment wires. The use of a plurality of treatment wires at different heights at which the sensor element or the position marker is attached provides additional stability with respect to a rotational movement of the sensor element or of the position marker about the treatment wire.

A first end of a supporting element can, for example, be detachably attached to the dental brackets, wherein a second end of the supporting element is detachably attached to the sensor element or to the position marker. This two-part configuration of the part of the measurement system that is attached to the patient's lower jaw advantageously offers the possibility that the sensor element or the position marker be attached only in a final step after the fixing of the supporting element to the patient's lower jaw is complete. This facilitates the attachment or the alignment and, for example, also prevents the sensor element or the position marker from being damaged during the attachment process.

A further aspect of the present invention provides a measurement apparatus for recording jaw movements of the patient according to one of the previously-described methods, the measurement apparatus comprising a measurement system having a sensor element and a position marker, and dental brackets which are attached on one side to the teeth of the lower jaw using a soluble adhesive and which have a receptacle on the other side, wherein the sensor element or the position marker is detachably attachable.

The sensor element or the position marker can, for example, be detachably attached to the receptacles of the dental brackets via a fitting counterpart of the receptacles. It is hereby advantageously possible to insert the sensor element or the position marker in the receptacle in a mechanically fixed manner without using additional adhesive means and to provide that the sensor element or the position marker can again be mechanically detached from the receptacle by performing specific work steps. The corresponding components are hereby separated from one another in a destruction-free manner, with the result that they are reusable after disinfection.

The treatment wire can, for example, be detachably fixed in the receptacles and extend horizontally between two respective dental brackets, wherein the sensor element or the position marker is detachably attached to the treatment wire via wire brackets. The wire brackets make it possible here advantageously to fix the sensor element or the position marker to the treatment wires of already existing braces. The wire brackets can, for example, be clipped, latched, screwed or attached in another way to the treatment wire.

A further aspect of the present invention provides for the use of the present invention for orthodontic treatment on the teeth of the patient's lower jaw, wherein a detachably-supported dental bracket is used for attachment of a measurement apparatus. Conventional braces can thus advantageously be used for attaching the measurement system. It is thereby possible, depending on the circumstances, to use braces which have already been applied to the patient or to attach the braces specifically for the measurement apparatus.

The sensor element or the position marker can, for example, be detachably attached to the dental brackets or the wire brackets via a supporting element.

The present invention will be explained in greater detail below under reference to the drawings on the basis of the exemplary embodiments.

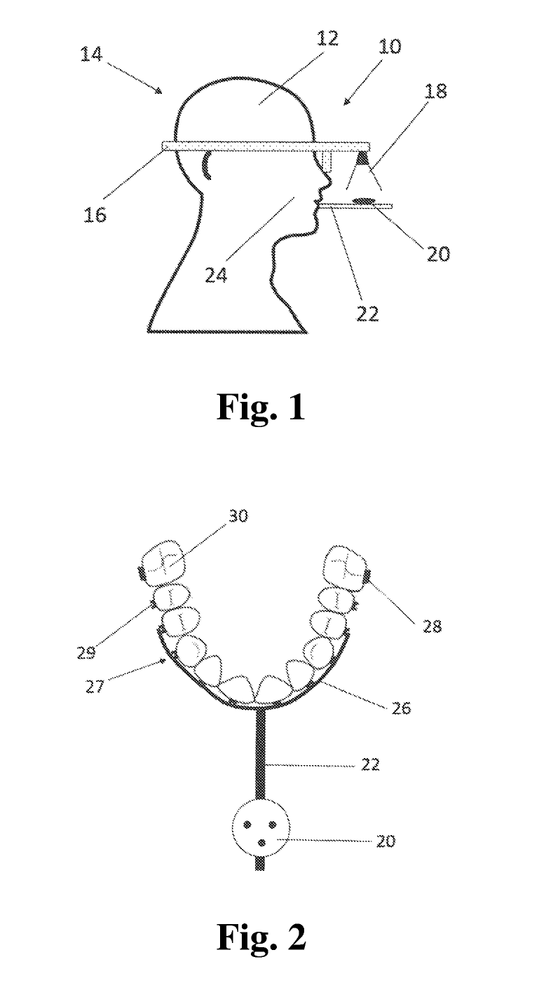

FIG. 1 shows the manner of attachment of the measurement system 10 of the measurement apparatus on the head 12 of a patient 14. The measurement system 10 in the present case has a two-part design and includes a sensor element 18 and a position marker 20, wherein the position marker 20 is placed onto a supporting element 22 which is inserted at least partially into the oral cavity 24 of the patient 14 and is here attached to the teeth 30 of the lower jaw of the patient 14. The sensor element 18 of the measurement system 10 is attached to the head 12 of the patient 14 via a bracket holder 16 and has as little movement play as possible, with the result that it is possible to deduce the jaw movement of the patient 14 from the relative movement between the position marker 20 and the sensor element 18 and to minimize erroneous influences.

The measurement system 10 can expediently also be designed as a single piece, wherein in this case the supporting element 22 and the bracket holder 16 can, for example, be connected by way of an articulation. The position marker 20 and the supporting element 22 can likewise be present in the form of a position marker 20 which is designed as one piece. The two-part configuration offers the advantage that the position marker 20 can still be displaced and correspondingly finely aligned after the supporting element 22 has been attached to the teeth 30 of the patient 14. If the patient 14 then moves his/her lower jaw, the position marker 20 follows this movement, wherein the resulting movement curve is recorded by the sensor element 18 and stored in a computer for later evaluation.

FIG. 2 shows a first method according to the present invention for attaching the position marker 20 of the measurement apparatus to the teeth 30 of the lower jaw of the patient 14. Dental brackets 28 are here adhesively bonded by the dentist to the vestibular surfaces of the teeth 30 on a first side at selected attachment points 27. The vestibular surfaces refer to the surface sides of the teeth 30 that face the oral cavity of the patient 14. On a second side of the dental brackets 28 which face away from the teeth 30, receptacles 29 are provided so as to be able to attach other components to the dental brackets 28. FIG. 2 shows a coupling element 26 which has fitting receptacle counterparts and which is mechanically attached to the receptacles 29 via the receptacle counterparts by the dentist. The coupling element 26 is here mechanically fixed in the receptacles 29 via previously defined work steps and without adhesive and can be removed again in a destruction-free manner by subsequent work steps after the chewing movement has been recorded.

The coupling element 26 can be easily bent by the dentist so that the outer shape of the coupling unit 26 corresponds to that of the dental arch of the patient 14. The attachment points 27 in FIG. 2 are arranged mirror symmetrically with respect to the mirror-symmetric axis through the lower jaw. The supporting element 22 is fixedly attached by its one end to the coupling element 26, wherein the position marker 20 is placed on the end of the supporting element 22 that faces away from the teeth 30, with the result that the movement of the position marker 20 is coupled to the movement of the lower jaw. The coupling unit 26 in FIG. 2 is consequently fixed securely to the teeth 30 of the patient 14 so that even greater torques, as occur, for example, due to the jaw movement of the patient 14, do not cause a jiggling or displacement of the position marker 20 and the chewing movements are precisely recorded.

The attachment points 27, the shape of the dental brackets 28, and the receptacles 29 can be determined or specified via imaging methods so that the receptacle counterparts and the coupling element 26 can accordingly be appropriately produced in advance. The coupling element 26 is produced, for example, via a rapid prototyping method, milled or printed, so that it fits in a form-fitting and/or force-fitting manner.

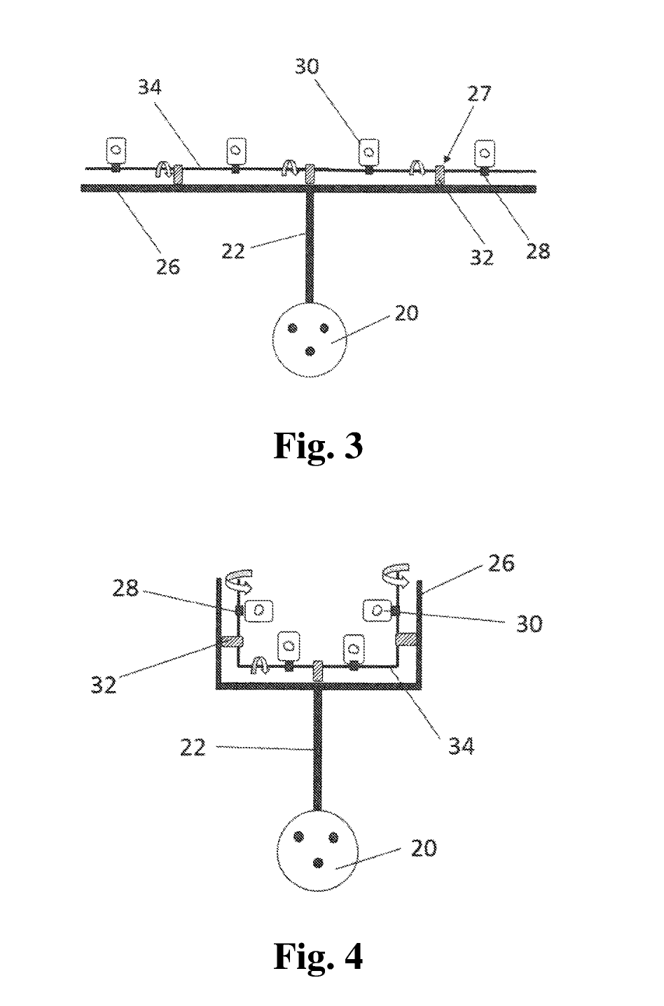

FIG. 3 shows a first schematic configuration of the attachment of the position marker 20 to the coupling element 26 of braces for illustration purposes. The coupling element 26 in this configuration is not directly connected to the dental brackets 28 of the teeth 30, but via wire brackets 32, which are attached to a treatment wire 34 that extends through the dental brackets 28 and consequently connects them. One known example of such an arrangement in dental practice is a conventional set of braces. The treatment wire 34 is mechanically fixed to the dental brackets 28, wherein the wire brackets 32, which are fixedly connected to the coupling element 26 by their one end, are mechanically fixed to the treatment wire 34 by their other end. The wire brackets 32 can here expediently be clipped, latched or screwed to the treatment wire 34 and be easily removed again later. In this first configuration, the treatment wire 34 defines a single axis of rotation by way of its rectilinear extent. The result is that a torque which acts on the supporting element 22 or the position marker 20 easily leads to a twisting of the position marker 22 in relation to said axis of rotation.

FIG. 4 shows how a total of three axes of rotation are formed by a rectangular progression of the one-piece treatment wire 34 which block one another with respect to their respective rotation due to the one-piece configuration of the treatment wire 34 in a manner so that a torque acting on the supporting element 22 does not result in a rotation of the supporting element 22. The position marker 20 is consequently fixed even in the case of an indirect attachment to the dental brackets 28 via the treatment wire 34 and the wire brackets 32 so that the jaw movements can be recorded precisely.

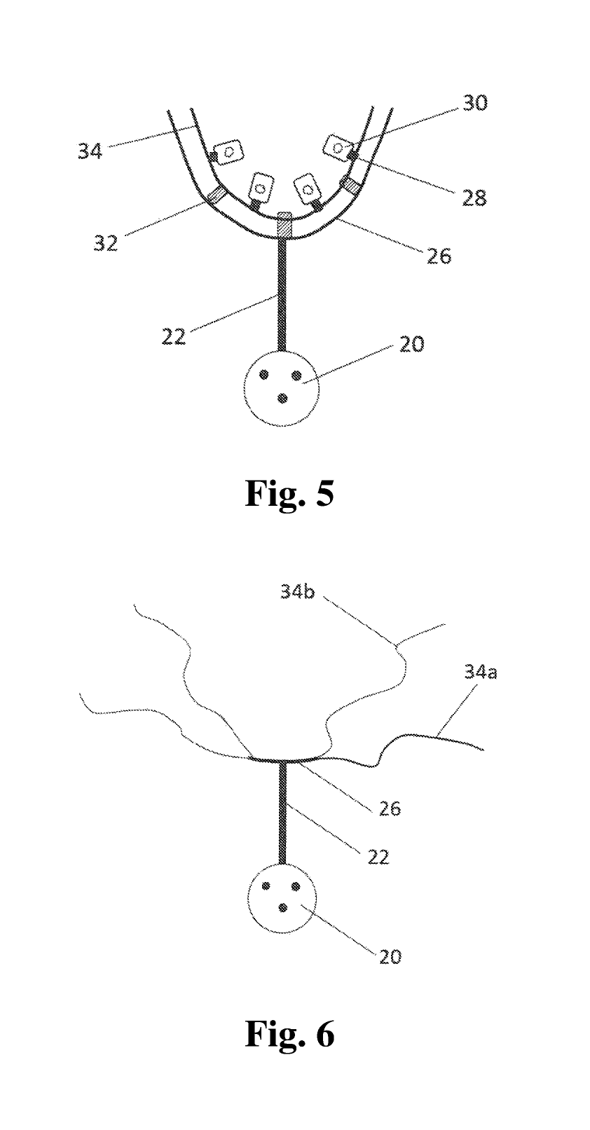

FIG. 5 shows a second method according to the present invention for attaching the position marker 20 to braces of the patient 14 via the treatment wire 34. Geometrically speaking, FIG. 5 is situated between the extreme cases of FIG. 3 and FIG. 4 which were used for illustration purposes. The curvature of the coupling element 26 and of the treatment wire 34, which was adapted to the dental arch, in the manner of FIG. 4, has the result that the position marker 20 is fixed despite acting torques to the lower jaw of the patient 14 and does not rotate.

FIG. 6 shows a plurality of treatment wires 34a, 34b which are attached to a coupling element 26 and which extend away therefrom. The use of a plurality of treatment wires 34a, 34b instead of a single treatment wire 34 makes an even stronger attachment of the position marker 20 to the lower jaw of the patient 14 possible. The treating dentist decides whether it is necessary in the individual case to use of a plurality of treatment wires 34a, 34b in the case of a specific patient 14.

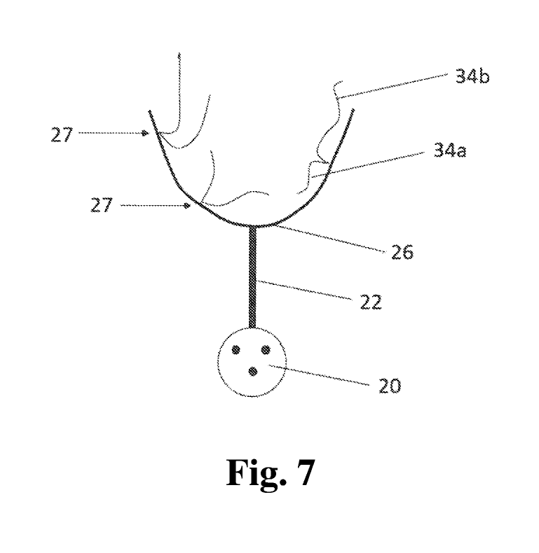

In addition to the plurality of treatment wires 34a, 34b, FIG. 7 shows a freely selectable configuration of the attachment points 27 on the coupling element 26. This is necessary under certain circumstances if the patient 14 has a diseased teeth 30 on which no torque should act in order to preserve the diseased tooth 30, or, if the patient 14 is missing teeth, so that no dental brackets 28 are attachable to these locations. The dentist decides the selection of the attachment points 27 based on the individual patient 14 in order to provide a sufficient attachment of the position marker 20 to the lower jaw of the patient 14.

The present invention is not limited to embodiments described herein; reference should be had to the appended claims.

What is claimed is:

1-11. (canceled)

12. A method for recording jaw movements of a patient, the method comprising:
detachably adhering dental brackets to teeth surfaces of the teeth of a lower jaw of the patient;
attaching a position marker and a sensor element which is configured to detect the position marker to a head of the patient,
wherein,
the sensor element is attached to a skull of the patient, and the position marker is attached to the lower jaw of the patient via the dental brackets, or
the sensor element is attached to the lower jaw of the patient via the dental brackets, and the position marker is attached to the skull of the patient; and
recording, via a jaw movement, a relative movement of the sensor element relative to the position marker.

13. The method as recited in claim 12, wherein,
the dental brackets comprise attachment points, and
the method further comprises:
detachably attaching the sensor element or the position marker to the attachment points of the dental brackets.

14. The method as recited in claim 13, wherein the attachment points are attached to the lower jaw of the patient so as to be mirror-symmetric.

15. The method as recited in claim 12, further comprising:
detachably attaching a treatment wire to the dental brackets so that two respective dental brackets are horizontally connected via the treatment wire; and
detachably attaching the sensor element or the position marker between two dental brackets via the treatment wire.

16. The method as recited in claim 15, further comprising:
arranging a plurality of treatment wires to extend through the dental brackets; and
attaching the sensor element or the position marker to each of the plurality of treatment wires.

17. The method as recited in claim 12, further comprising:
providing a supporting element comprising a first end and a second end;
detachably arranging the first end of the supporting element to the dental brackets; and
detachably arranging the second end of the supporting element to the sensor element or to the position marker.

18. A measurement apparatus for recording the jaw movements of the patient as recited in claim 12, the measurement apparatus comprising:
a measurement system comprising a sensor element and a position marker,
wherein,
dental brackets are detachably attached to the teeth of the lower jaw of the patient using an adhesive, the dental brackets each comprising a receptacle which are configured to have the sensor element or the position marker be detachably attachable thereto.

19. The measurement apparatus as recited in claim 18, wherein,
the sensor element or the position marker comprise a receptacle counterpart; and
the sensor element or the position marker is detachably attached to the receptacles of the dental brackets via the receptacle counterpart.

20. The measurement apparatus as recited in claim 18, further comprising:
a treatment wire which is configured to be detachably fixed in the receptacles and to extend horizontally between the dental brackets; and
wire brackets,
wherein,
the sensor element or the position marker is detachably attached to the treatment wire via the wire brackets.

21. The measurement apparatus as recited in claim 20, further comprising:
a supporting element,
wherein,
the sensor element or the position marker is detachably connected to the dental brackets or to the treatment wire via the supporting element.

22. A method of using a dental bracket for an orthodontic treatment, the method comprising:
detachably providing the dental bracket on the teeth of a lower jaw of a patient;
attaching the measurement apparatus as recited in claim 18 to the dental bracket; and
using the dental bracket to provide the orthodontic treatment.
